(19) United States
(12) Patent Application Publication
(10) Pub. No.: US 2015/0315247 A1
(43) Pub. Date: Nov. 5, 2015

(54) High-affinity peptide-based anticancer vaccination to overcome tumor resistance to immunostimulatory antibodies and to identify TCRs that can be used successfully in adoptive T cell therapy

(71) Applicants: David Charles Binder, Burr Ridge, IL (US); Hans Schreiber, Chicago, IL (US)

(72) Inventors: David Charles Binder, Burr Ridge, IL (US); Hans Schreiber, Chicago, IL (US)

(73) Assignee: David Charles Binder, Burr Ridge, IL (US)

(21) Appl. No.: 14/269,180

(22) Filed: May 4, 2014

(51) Int. Cl. C07K 14/255; C07K 14/465; A61K 39/395; C07K 14/005

(52) U.S. Cl. CPC C07K 14/255; C07K 14/005; C07K 14/465; A61K 39/39558; C07K 2319/33

(57) ABSTRACT

The invention provides a methodology to use cancer vaccination to overcome tumor resistance to immunostimulatory antibodies. Our cancer vaccination approach relies on targeting mutant tumor-specific peptides that have high affinity for the major histocompatibility complex. Treatment with bacteria expressing high affinity tumor-specific peptides combined with anti-PD-L1 monoclonal antibody can eradicate long-established tumors resistant to anti-PD-L1 and anti-CTLA-4 alone. In addition, we discovered that adoptive transfer of T cells with this same vaccine-generated T cell receptor specificity can also eradicate long-established tumors. These results demonstrate that cancer vaccination approaches should target peptides with high peptide-MHC affinity to (i) overcome tumor resistance to immunostimulatory antibodies and (ii) identify T cell receptors that can be used successfully for adoptive T cell transfer.

### FIELD OF INVENTION

The invention relates to the field of tumor immunotherapy: therapeutic vaccines, immunostimulatory antibodies, and adoptive T cell transfer.

### BACKGROUND OF INVENTION

Tumors can escape immune control despite expressing tumor-specific antigens arising from mutations. While immunogenic cancer cells can induce functional CD8⁺ T cell responses, this is usually restricted to early stages of tumor growth. Immunity rapidly decays with tumor growth, concurrent with the establishment of a tumor microenvironment in which cancer cells are embedded in a suppressive tumor stroma. Human tumors are regularly infiltrated by dysfunctional endogenous PD-1-expressing CD8⁺ T cells. Proliferation and effector function of these CD8⁺ T cells are likely impaired due to engagement of PD-1 with PD-L1 expressed by cancer cells and/or antigen-presenting cells (APCs).

Many clinical studies are trying to rescue the function of T cells against immunogenic tumors. Monoclonal antibodies that block immunosuppressive T-cell receptors such as programmed cell death 1 (PCD1, best known as PD-1) and cytotoxic lymphocyte-associated protein 4 (CTLA-4) elicit strong therapeutic responses in some patients; an effect that seems to be durable with anti-PD-1 since many tumors that responded to therapy did not relapse within the 1st year after treatment initiation. However, the majority of cancer patients, including individuals with signs of a pre-existing T-cell response do not respond to these antibodies.

The current clinical challenge is therefore to develop a strategy to rescue T-cell responses in patients that are resistant to immunostimulatory antibodies. No existing tumor immunotherapeutic approach, including previously described therapeutic vaccination protocols, is able to eradicate long-established preclinical tumors or clinical-tumors that are resistant to immunostimulatory antibodies.

While any approach that can rescue dysfunctional endogenous T cells in tumors would have great clinical value, a second approach with strong clinical potential is adoptive T cell transfer. Adoptive transfer involves expanding tumor-reactive T cells in vitro prior to infusing these activated cells into patients. One adoptive transfer approach involves isolating tumor-infiltrating lymphocytes (TILs) from a patient, expanding these TILS in vitro with IL-2, and reinfusing these cells into a lymphodepleted patient. As a second adoptive transfer approach, a patient's autologous T cells are transduced with a T cell receptor. These transduced T cells are then transferred into a lymphodepleted patient.

Adoptive transfer of autologous tumor-infiltrating lymphocytes after expansion and re-stimulation in culture can cause complete responses in 10% of patients. Responses in this small percentage of patients correlate with patients having tumor-infiltrating lymphocytes that respond to autologous tumor-specific (mutant) peptides that bind to human leukocyte antigen molecules (human major histocompatibility complex (MHC)) with highest (low nanomolar or sub-nanomolar affinity) affinity. Despite the promise using adoptive transfer, there is no approach to identify T cell receptors that can recognize mutant tumor-specific peptides. Identifying these specific mutant T cell receptors is the biggest challenge for adoptive transfer to become clinically feasible.

While tumor-specific peptides remain powerful targets for T cells to eradicate tumors, only recently has new technology allowed characterization of tumor-specific peptides on human tumors. Determining the set of mutations in any given cancer has become fast and affordable with genomic exome sequencing. It is also now possible to determine the affinity of the mutant peptide to the MHC using algorithms or cell-based based assays.

Using a clinically-relevant animal tumor model is essential to meaningfully test the efficacy of new tumor immunotherapeutic approaches. Most tumor studies involve injecting syngeneic cancer cells subcutaneously on the backs of mice. The majority of these tumor studies treat “tumors” only a few days following cancer cell inoculation in mice. These “tumors” are very small and histologically resemble acute inflammatory lesions, rather than tumors. These early “tumors” lack immunosuppression, a fibroblastic stroma, and blood vessels that contribute to tumor aggressiveness. However, once tumors become long-established (defined here as z 2 weeks-old and exceeding 100 mm³), they resemble clinical tumors histologically and are very resistant to immunotherapy. Therefore, when testing new tumor immunotherapies, it is essential to treat long-established preclinical tumors since it is only after 2 weeks that artifacts from cancer cell inoculation-significant necrosis, acute inflammation, and an initial functional T cell response-finally resolve. Previous cancer vaccination and immunostimulatory approaches have only reported successful results when treating “early” tumors, but fail to report success treating the clinically relevant long-established experimental tumors.

### BRIEF SUMMARY OF THE INVENTION

The invention represents a significant breakthrough in cancer therapy as it is the first method to use cancer vaccination to overcome tumor resistance to immunostimulatory antibodies. The success of this cancer vaccination approach was achieved in a long-established clinically-relevant tumor model.

Our invention is to overcome tumor resistance to immunostimulatory antibodies using bacteria that deliver exogenous tumor-specific peptide with high peptide-MHC affinity. The model bacterium that we used was Salmonella Typhimurium A1-R. Our vaccination approach targeted a model mutant tumor-specific peptide with high peptide-MHC affinity. We treated aggressive long-established melanoma tumors in immunocompetent mice that were infiltrated by dysfunctional endogenous tumor-specific CD8 T cells. These tumors resemble the aggressive and immunosuppressive tumors seen in cancer patients.

Treatment with bacteria producing tumor-specific peptide led to eradication of long-, established tumors in 31% of mice. Combining this bacterial vaccine with anti-PD-L1 led to tumor eradication in 80% of mice. Importantly, these tumors did not respond to treatment with immunostimulatory anti-PD-L1 and anti-CTLA-4 antibodies alone.

When T cells with the same T cell receptor specificity, as generated by vaccination, were used for adoptive T cell transfer, tumors were eradicated in 100% of mice.

This is the first cancer vaccination approach to (i) eradicate advanced long-established tumors (>100 mm̂3 and >14 days post-inoculation) as a monotherapy, (ii) synergize with anti-PD-L1 to consistently eradicate tumors, (iii) identify T cell receptors that can be successfully used for adoptive T cell transfer, and (iv) successfully eradicate long-established tumors by vaccinating with a tumor-specific peptide that has high peptide-MHC affinity. The success of this approach was achieved using long-established tumors that did not respond to immunostimulatory antibodies alone.

### BRIEF DESCRIPTION OF THE DRAWINGS

FIG. 1 is a diagram showing our bacterial tumor-specific peptide expression system. It also demonstrates that bacteria can deliver tumor-specific peptide into antigen-presenting cells for MHC presentation to CD8 T cells.

A) Diagram of the SIINF, SNFV, and EGFP constructs used in this study. B) High-copy number plasmids encoding the respective SIINF and SNFV fusion protein constructs were introduced into the A1-R strain. Whole bacterial lysates were examined for fusion protein expression using the anti-M45 antibody. C) J774 K^{b}-expressing macrophages were infected with A1-R SIINF or A1-R SNFV. Infected macrophages were then incubated with the B3Z (SIINFEKL-specific CD8⁺ T cell) hybridoma for 24 hours. B3Z stimulation was evaluated by the amount of IL-2 secreted into the culture as determined by ELISA. Data are representative of 2 independent experiments.

FIG. 2 is a diagram showing that long-established melanomas contain dysfunctional endogenous tumor-specific CD8 T cells.

A) C57BL/6 mice were inoculated with B16-OVA cancer cells. At the indicated times, the peripheral blood leukocytes were stained with anti-CD8 and either SIINF/K^{b}-dimerX or control SIYR/K^{b}-dimerX. Data were pooled from 6 total mice with progressively growing tumors compiled from 2 independent experiments. B) B16-OVA and B16 tumor growth was measured following injection of 5×10⁶ cancer cells into C57BL/6 or C57BL/6 CD8^{-/-} mice. The mean tumor volume (±SD) was calculated for each time-point. Each group consisted of 4 mice with progressively growing tumors; data are representative of 2 independent experiments. C) B16-OVA cancer cells were inoculated in a C57BL/6 mouse. 18 days later, the B16-OVA tumor reached 144 mm³. Single cell suspensions were made from the tumor-draining lymph node (TDLN), lungs, spleen, and tumor. The suspensions were stained with anti-CD8 and either SIINF/K^{b}-dimerX (SIINF-dX) or control SIYR/K^{b}-dimerX (SIYR-dX). Data are representative of 3 total mice from 2 independent experiments.

FIG. 3 is a diagram showing that bacteria producing tumor-specific peptide can rescue the tumor-specific CD8 T cell response in the blood and tumors of mice bearing long-established tumors.

C57BL/6 mice bearing B16-OVA tumors were analyzed from the following groups: untreated tumors at day 0 (defined when tumors reached 100-168 mm³); untreated tumors at day 8; A1-R control-treated tumors 8-9 days post-treatment; and A1-R SIINF-treated tumors 8-9 days post-treatment. A) The peripheral blood was stained with anti-CD8 and SIINF/K^{b}-dimerX. The top panel is a representative stain from one mouse and the bottom panel contains pooled data with each mouse represented by a single dot.

The A1-R control treatment group consists of 2 mice treated with A1-R SNFV and 2 mice treated with A1-R EGFP. ***p<0.001 when comparing A1-R SIINF to each other group. B) The upper two rows analyzed the percentage of SIINF-specific CD8⁺ T cells from tumors. Single cell suspensions from tumors were stained with anti-CD8 and either SIINF/K^{b}-dimerX or control SIYR/K^{b}-dimerX. The top panel is a representative stain from one mouse per group. The bottom panel contains pooled data from individual mice derived from at least 2 independent experiments per treatment group. The percentage of SIINF-specific CD8⁺ T cells represents the percentage of cells that stained positive with SIINF/K^{b}-dimerX subtracted by the background percentage of cells that stained positive with SIYR/K^{b}-dimerX. The difference between the A1-R SIINF group and each other group was non-significant (n.s.). The lower three rows analyzed cytokine production by SIINF-specific CD8⁺ T cells from the tumor. The same tumor cell suspensions, as analyzed in the upper two rows, were restimulated with SIINF peptide in the presence of Brefeldin A for 5 hrs. Cells were stained with SIINF/K^{b}-dimerX or control SIYR/K^{b}-dimerX, anti-CD8, anti-IFN-γ, and anti-TNF-α. Top panel shows a representative anti-IFN-γ and anti-TNF-α stain from gated SIINF/K^{b}-dimerX⁺ CD8⁺ double-positive cells. The bottom panels show pooled data. The percentage of IFN-γ⁺ or IFN-γ⁺ TNF-α⁺ cells was defined as the percentage of SIINF-specific CD8⁺ T cells that stained positive with anti-IFN-γ and/or anti-TNF-α compared to the isotype controls.

The A1-14 control treatment group consisted of 4 mice treated with A1-R SNFV. ***p≦0.001 when comparing A1-R SIINF to each other group. **p≦0.002 when comparing A1-R OVA to each other group.

FIG. 4 is a diagram showing that treatment with bacteria producing tumor-specific peptide can eradicate tumors in 31% of mice bearing long-established tumors. This effect is CD8 T cell dependent. The treatment effect of bacteria alone is minimal. C57BL/6 mice, bearing established B16-OVA tumors were left untreated; treated one time with A1-R control; treated one time with A1-R SIINF; or treated one time with A1-R SIINF followed by treatment with the αCD8 depletion antibody. Lines indicate individual mice. The 4 mice that rejected the tumor following A1-R SIINF treatment were held for at least 100 days post-treatment. To determine if the tumors were eradicated, 3 of these mice were injected with 3 doses of 200 μg αCD8 at 3 day intervals. There was no tumor outgrowth for the 34 days following CD8⁺ T cell depletion before the mice were sacrificed. The A1-R control treatment group consisted of 3 mice treated with A1-R EGFP and 2 mice treated with A1-R SNFV. [symbol] indicates a single mouse that died during the experiment.

FIG. 5 is a diagram showing that weekly treatment with bacteria producing tumor-specific peptide can eradicate tumors in 31% of long-established tumor-bearing mice. C57BL/6 mice bearing established B16-OVA tumors were treated weekly with A1-R control or A1-R SIINF. There was a 5 day interval between the 1st and 2nd bacterial injections and −7 day interval between all other injections. Mice were continuously treated until tumors exceeded 1.5 cm³ or tumors were fully rejected. Lines indicate single mice. Three mice fully rejected B16-OVA following weekly A1-R SIINF treatment. One of these mice was injected with 3 doses of 200 μg αCD8 depletion antibody at 3 day intervals. This mouse fully eradicated the B16-OVA tumor as there was no tumor outgrowth for the 34 days following CD8⁺ T cell depletion before the mouse was sacrificed. The A1-R control treatment group consisted of 4 mice treated with A1-R EGFP and 6 mice treated with A1-R SNFV. Cross indicates mouse that died during the experiment.

FIG. 6 is a diagram showing that tumor-localized CD8 T cells express a high level of PD-1 prior to treatment as well as following treatment with bacteria producing tumor-specific peptide.

Single cell suspensions, derived from the tumor or spleen, were stained with anti-CD8, SIINF/K^{b}-dimerX or SIYR/K^{b}-dimerX, and anti-PD-1. PD-1 expression, on gated SIINF/K^{b}-dimerX⁺ CD8⁺ double-positive cells, was analyzed from B16-OVA tumor-bearing C57BL/6 mice in the indicated groups: untreated at day 0 (defined as when tumors were between 100-168 mm³); untreated at day 8; A1-R control-treated at 8-9 days post-treatment; and A1-R SIINF-treated at 8-9 days post-treatment. Representative stain is shown in the top panel and pooled data from individual mice were compiled in the bottom panel. Data were pooled from at least 2 independent experiments per group. The A1-R control treatment group consisted of 4 mice treated with A1-R SNFV. ****p<0.0001 when comparing the A1-R SIINF spleen group to the A1-R SIINF tumor group. There was no significant statistical difference when comparing PD-1 expression on SIINF-specific CD8⁺ T cells from the tumor between the different treatment groups.

FIG. 7 is a diagram showing that treatment with bacteria producing tumor-specific peptide synergizes with anti-PD-L1 to eradicate long-established tumors in 80% of mice. Tumors do not respond to anti-CTLA-4 and anti-PD-L1 treatment alone. Tumor eradication in mice treated with bacteria producing tumor-specific peptide and anti-PD-L1 correlates with enhanced CD8 T cell expansion in the peripheral blood. A) C57BL/6 mice bearing established B16-OVA tumors were treated as indicated: A blue dot represents the initial time of treatment with αCTLA-4 and αPD-L1 for an individual mouse. A red dot represents treatment with A1-R SIINF for an individual mouse. In each treatment group, mice were treated with 100 μg αCTLA-4 and/or 150 μg αPD-L1 every third day until the tumor relapsed completely (>1.5 cm³) or was rejected. Mice that rejected tumors were held for at least 100 days post-treatment prior to administration of 3 doses of 200 μg αCD8 at 3 day intervals. There was no tumor outgrowth for at least 40 days post-CD8⁺ T cell depletion before the mice were sacrificed, demonstrating that tumors were fully eradicated. B) At 8 or 9 days post-treatment, the peripheral blood from mice in the indicated treatment groups was stained with anti-CD8 and SIINF/K^{b}-dimerX. The percentage of SIINF-specific CD8⁺ T cells from mice that rejected B16-OVA versus relapsed following A1-R SIINF treatment was significantly different (p<0.01).

FIG. 8 is a table showing the compilation of tumor treatment studies.

FIG. 9 is a diagram showing that adoptively-transferred CD8 T cells, with the same T cell receptor specificity as generated by bacterial vaccination, eradicated tumors in 100% of mice.

B6 mice bearing established B16OVA tumors (day 18-24) were treated with preconditioning irradiation (450 rad) followed or not by splenocytes from 1 naïve OT1 mouse 24 later. Data are pooled from two independent experiments.

### DETAILED DESCRIPTION

Our invention is to overcome tumor resistance to immunostimulatory antibodies using bacteria that deliver exogenous tumor-specific peptide with high peptide-MHC affinity. Existing genomic exome sequencing technology of cancer, cells versus matched normal cells allows tumor-specific mutations to be identified. Existing cell-based assays and prediction algorithms can be used to determine which tumor-specific peptides have highest affinity for the MHC. To test the efficacy of treating tumors by targeting a peptide with high peptide-MHC affinity, we treated B16-OVA murine melanomas that expressed the model tumor-specific SIINFEKL peptide that has high peptide-H-2K^{b} (mouse MHC) affinity (IC₅₀ [nM]=0.9).

The model bacterium that we used was Salmonella Typhimurium A1-R. Salmonella Typhimurium A1-R was transformed with a plasmid encoding a fusion protein consisting of the first 104 amino acids of the Salmonella Typhimurium SopE gene, the M45 epitope from the adenovirus E4-6/7 protein, and amino acids 248-357 of ovalbumin. SopE targeted our fusion protein from the salmonella-containing vacuole into the cytosol of host cells where the process of MHC-loading of peptides begins. The M45 epitope was used to confirm fusion protein expression by Salmonella Typhimurium. The ovalbumin domain contained the immunodominant SIINFEKL epitope.

We treated long-established B16-OVA murine melanoma tumors, at least 100 mm³ and 14 days established, that expressed the SIINFEKL peptide. These tumors were infiltrated by dysfunctional endogenous tumor-specific CD8 T cells, thus resembling the aggressive and immunosuppressive tumors seen in cancer patients. Treatment with Salmonella Typhimurium A1-R producing SIINFEKL led to tumor eradication of long-established tumors in 31% of mice. Combining this bacterial vaccine with anti-PD-L1 led to tumor eradication in 80% of mice. Importantly, these tumors did not respond to the combination treatment of anti-PD-L1 and anti-CTLA-4.

We then tested if vaccination could be used to identify T cell receptors that could be successfully used for adoptive T cell therapy. Adoptively-transferred CD8 T cells, with the same T cell receptor specificity as generated by bacterial vaccination, eradicated tumors in 100% of mice.

These data are the first to demonstrate that (i) cancer vaccination can overcome tumor resistance to immunostimulatory antibodies and (ii) that vaccination can be used to identify T cell receptors that can be successfully used for adoptive T cell therapy.

### Example 1

### Bacteria Deliver Tumor-Specific Peptide to Antigen-Presenting Cells for MHC Presentation to CD8 T Cells

The following example shows that bacteria can be genetically modified to deliver tumor-specific peptide to antigen-presenting cells.

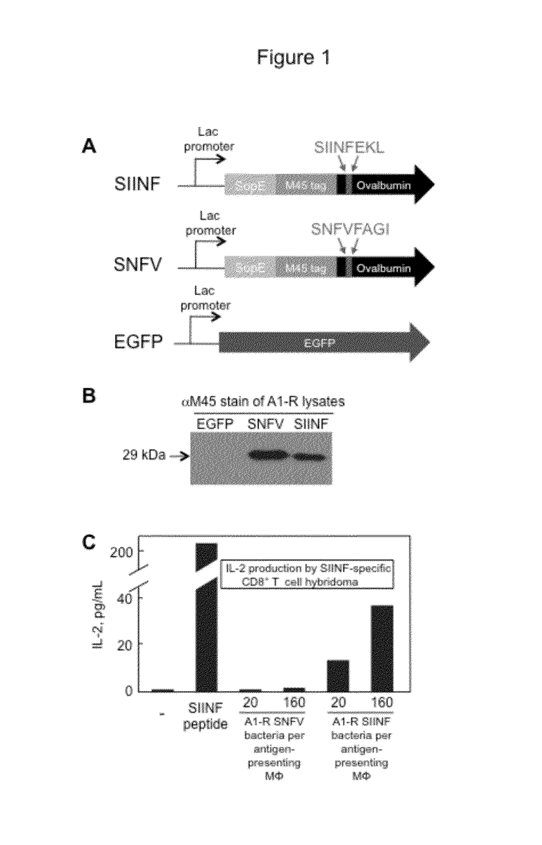

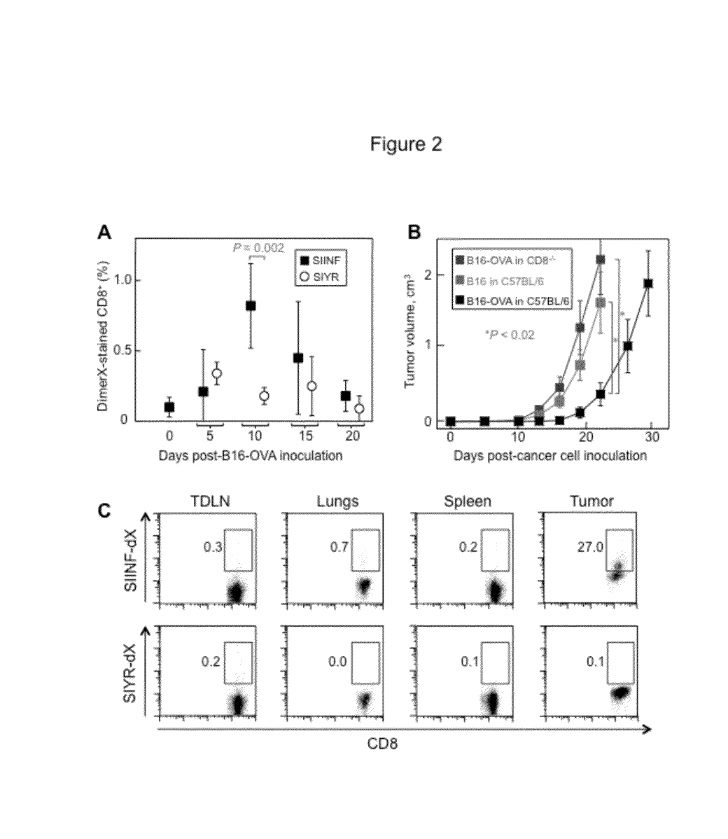

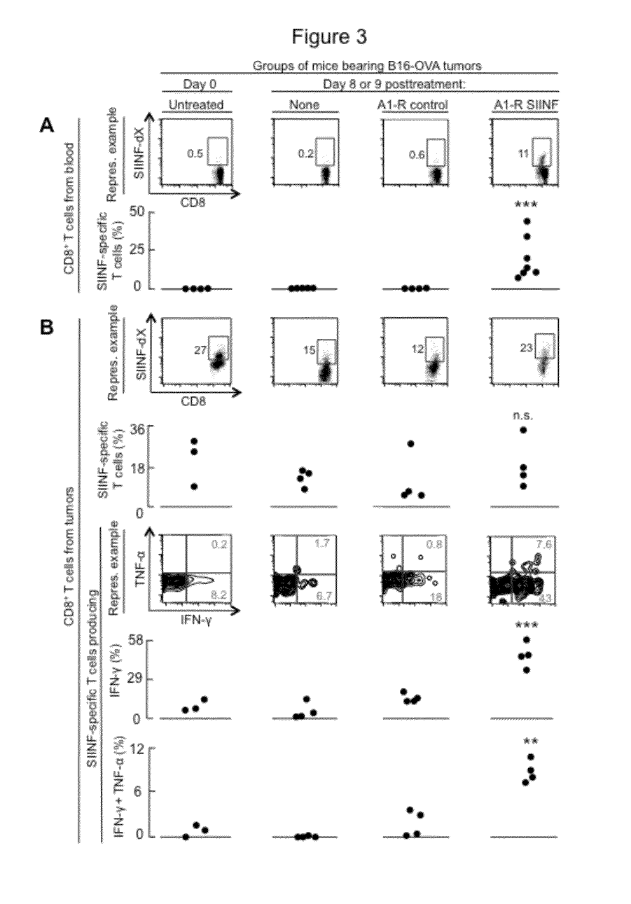

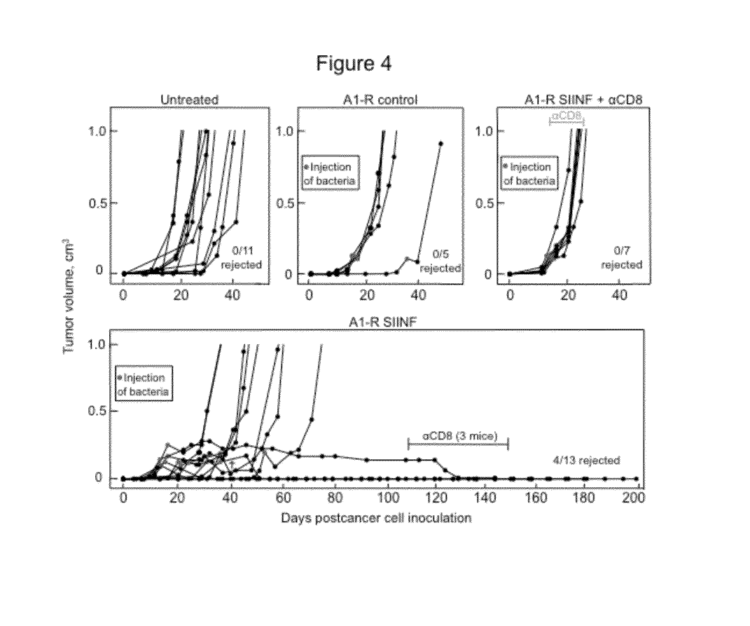

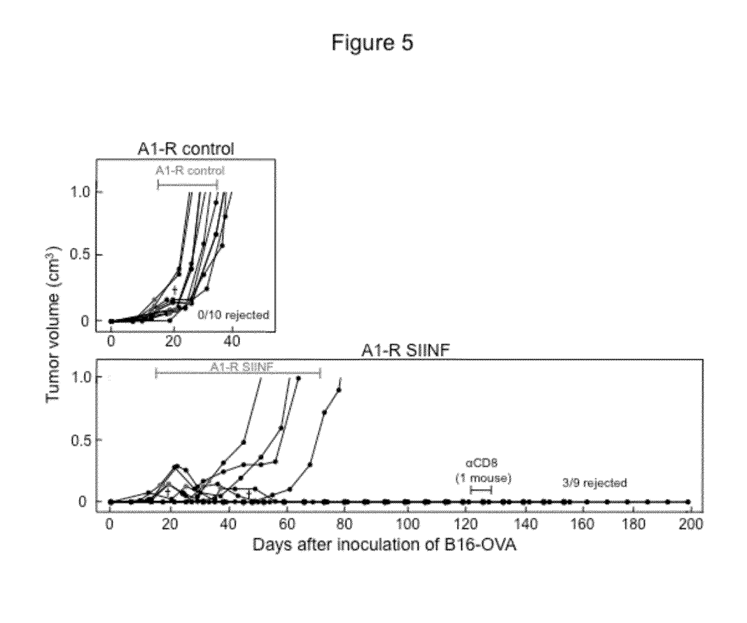

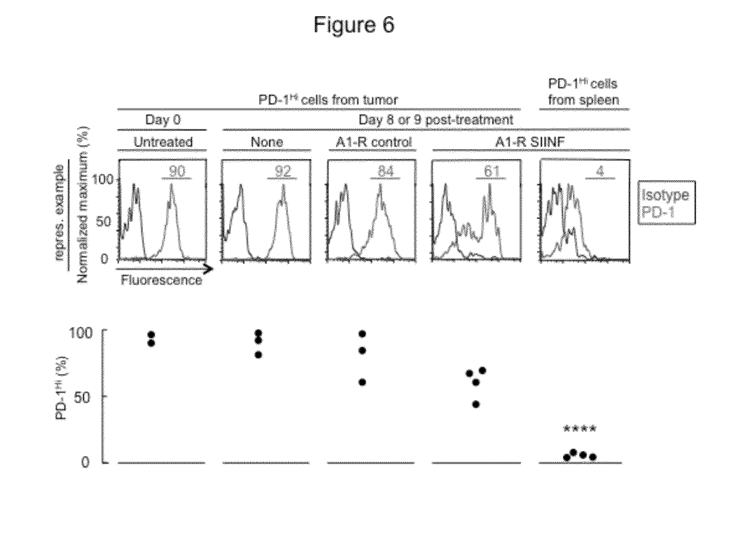

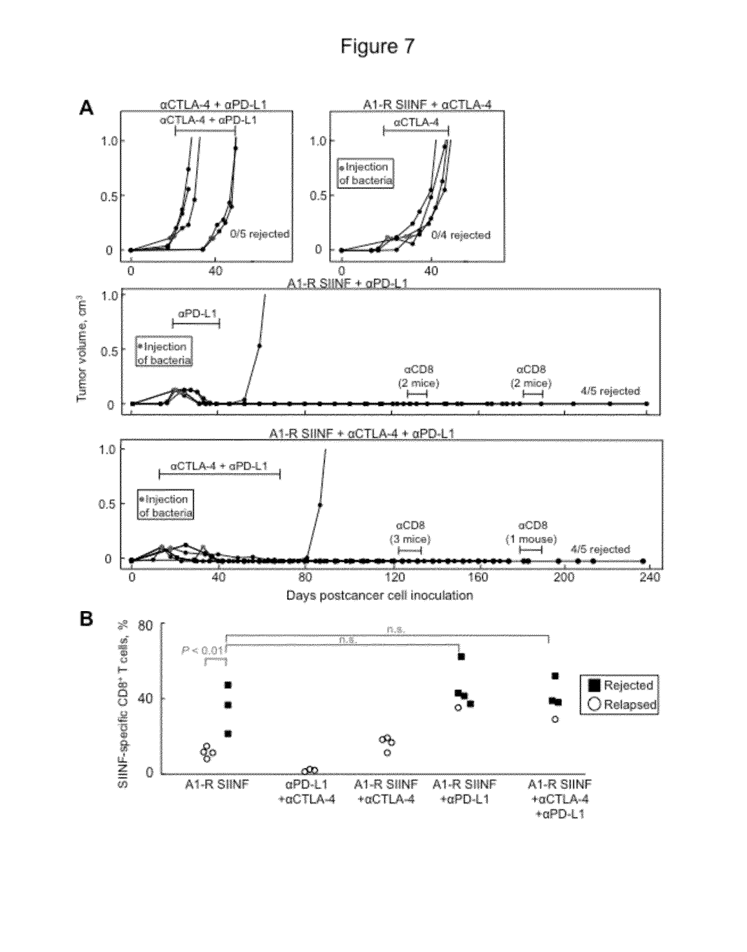

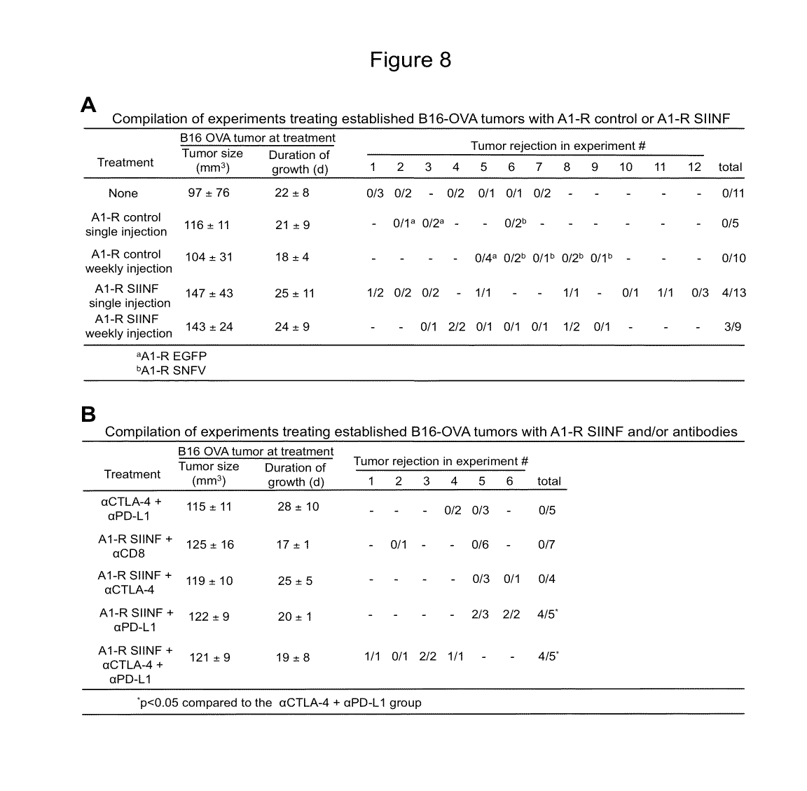

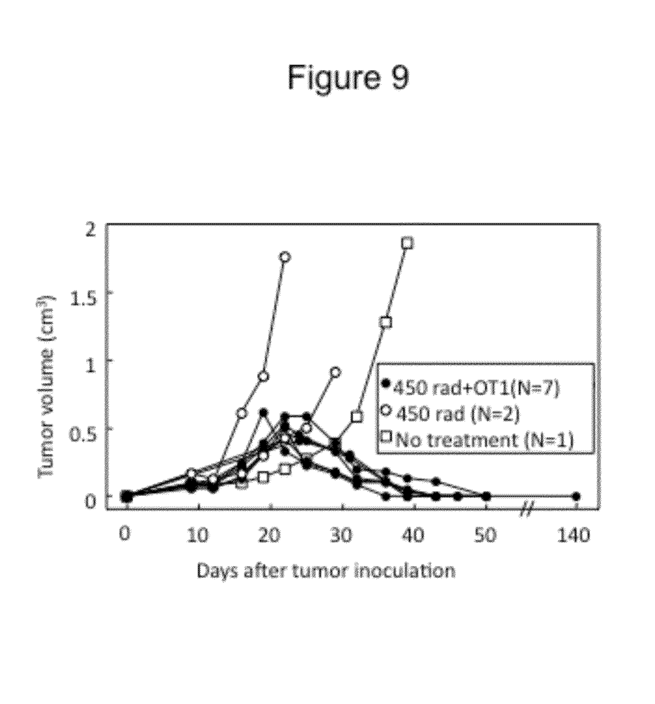

We constructed a fusion protein consisting of the SopE Type III secretion/translocation domain, an M45 epitope tag, and a SIINFEKL (SIINF)-containing OVA domain (amino acids 248-357). As SIINF-negative controls, we used (i) a similar fusion protein in which the irrelevant SNFVFAGI (SNFV) epitope (31) replaced the SIINF epitope or (ii) the enhanced green fluorescent protein (EGFP) (Diagrams in FIG. 1A).

The respective expression plasmids encoding fusion proteins were introduced into S. Typhimurium A1-R generating A1-R SIINF and A1-R SNFV respectively. A1-R EGFP has been previously described. Western blot verified that A1-R SIINF and A1-R SNFV expressed fusion protein (FIG. 1B).

We evaluated whether A1-R SIINF can deliver the SIINF epitope into APCs for correct MHC-I processing and presentation to CD8⁺ T cells. J774 K^{b}-expressing macrophages were infected in vitro with A1-R SIINF or A1-R SNFV. The capacity of the infected macrophages to present the SIINF epitope to CD8⁺ T cells was determined by stimulation of B3Z, a CD8⁺ T cell hybridoma specific for SIINF. A1-R SIINF- but not A1-R SNFV-infected macrophages stimulated B3Z to secrete IL-2 (FIG. 1C), demonstrating that A1-R SIINF can deliver the SIINF epitope into APCs for MHC-I presentation.

### Example 2

### Long-Established B16-OVA Tumors Resemble Clinical Tumors that are Also Heavily Infiltrated by Dysfunctional Endogenous Tumor-Specific CD8 T Cells

The following example shows that the tumors treated in this study resembled clinical tumors that are also immunosuppressive and infiltrated by dysfunctional tumor-specific CD8 T cells.

We evaluated the endogenous SIINF-specific CD8⁺ T cell response to B16-OVA cancer cell inoculation. At 10 days post-B16-OVA inoculation, the peripheral blood of C57BL/6 mice contained a population of SIINF-specific CD8⁺ T cells (FIG. 2A). B16-OVA outgrowth in wild-type mice was delayed compared to both parental B16 in wild-type mice and B16-OVA in CD8^{-/-} mice (FIG. 2B), suggesting that SIINF-specific CD8⁺ T cells delayed the outgrowth of B16-OVA tumors.

Despite this initial SIINF-specific CD8⁺ T cell response, B16-OVA tumors grew progressively and killed the host. As tumors reached 100-168 mm³, SIINF-specific CD8⁺ T cells reached a high percentage of total CD8⁺ T cells in the tumor (mean of 22%, ranging between 9-30%) but were at an undetectable or low percentage in the tumor-draining lymph node, spleen, and lungs (FIG. 2C).

### Example 3

### Bacteria Expressing Tumor-Specific Peptide Rescue the Dysfunctional Tumor-Specific CD8 T Cell Response in the Blood and Tumors of Mice Bearing Long-Established Tumors

The following example shows that bacterial vaccination with tumor-specific peptide can rescue the tumor-specific CD8 T cell response in mice bearing long-established tumors.

Mice bearing untreated B16-OVA tumors, established for at least 14 days and reaching 100-168 mm³, did not have detectable SIINF-specific CD8⁺ T cells in the lymph nodes, spleen, dr blood (FIGS. 2C and 3A). This group of mice was defined as “Day 0 untreated” since mice were treated throughout this study at this tumor size. Treating B16-OVA tumor-bearing mice with intravenously-injected A1-R SIINF induced systemic SIINF-specific CD8⁺ T cell proliferation in the lymphoid organs (Supplemental FIG. 2B) and generated a high percentage of SIINF-specific CD8⁺ T cells in the peripheral blood (FIG. 3A). To determine if this effect required A1-R to deliver SIINF, we used 2 independent SIINF-negative controls (A1-R SNFV and A1-R EGFP) collectively referred to as A1-R control. Mice treated with A1-R control did not have a high percentage of SIINF-specific CD8⁺ T cells in the peripheral blood (FIG. 3A), demonstrating that A1-R must deliver SIINF in order to rescue the SIINF-specific CD8⁺ T cell response in the periphery.

In mice bearing untreated B16-OVA tumors between 100-168 mm³, SIINF-specific CD8⁺ T cells reached a high percentage of total CD8⁺ T cells in the tumor (FIGS. 2C and 3B). However, these SIINF-specific CD8⁺ T cells were dysfunctional since they produced little IFN-γ and TNF-α upon peptide restimulation (FIG. 3B). Treatment with A1-R SIINF or A1-R control did not significantly change the percentage of SIINF-specific CD8⁺ T cells in the tumor (FIG. 3B). However, treatment with A1-R SIINF, but not A1-R control, rescued the capacity of SIINF-specific CD8⁺ T cells to produce IFN-γ and TNF-α (FIG. 3B). Thus, A1-R must produce SIINF in order to rescue SIINF-specific CD8⁺ T cell effector function in the tumor.

### Example 4

### Treatment with Bacteria Expressing Tumor-Specific Peptide Leads to Tumor Eradication in 32% of Mice Bearing Long-Established Tumors

The following example shows that bacterial vaccination with tumor-specific peptide can lead to eradication of long-established tumors.

Mice bearing long-established B16-OVA tumors were treated with a single injection of A1-R control, A1-R SIINF, or A1-R SIINF plus αCD8 depletion antibody (FIG. 4, FIG. 8). At 25 days post-treatment with a single A1-R injection, 11/13 mice treated with A1-R SIINF were alive compared to 0/5 mice treated with A1-R control (p=0.002) and 0/7 mice treated with A1-R SIINF plus αCD8 (p<0.0005). B16-OVA tumors were rejected in 4/13 mice treated with A1-R SIINF. We then tested if treating mice with weekly A1-R SIINF injections could maintain a stronger SIINF-specific CD8⁺ T cell response leading to more consistent tumor rejection (FIG. 5, FIG. 8). At 25 days post-treatment, 7/9 mice treated with A1-R SIINF were alive compared to 1/10 mice treated with A1-R control (p=0.005). Tumors were rejected in 3/9 mice treated with weekly A1-R SIINF. Therefore, A1-R SIINF treatment improves mouse survival by a SIINF-specific CD8⁺ T cell-dependent mechanism, and treatment with repeated doses does not provide a therapeutic benefit.

We tested whether tumors were fully eradicated in 4 mice that rejected the tumor. Three of the mice were originally treated with a single injection of A1-R SIINF and 1 mouse was originally treated with weekly injections of A1-R SIINF. All 4 mice were held for at least 90 days post-treatment and then depleted of CD8⁺ T cells. The tumors did not relapse (FIG. 4, FIG. 5), strongly suggesting that A1-R SIINF treatment completely eradicated B16-OVA tumors.

### Example 5

### Tumor-Localized CD8 T Cells Express a High Level of PD-1 Prior to Treatment as Well as Following Treatment with Bacteria Expressing Tumor-Specific Peptide

The following example strongly suggests that PD-1/PD-L1 interactions may limit the effectiveness of bacterial, vaccination, thereby suggesting combined use of anti-PD-L1 with bacterial vaccination.

Since CD8⁺ T cells can become dysfunctional through PD-1/PD-L1 interactions following chronic antigen exposure (11, 29, 36), we evaluated PD-1 expression before and after treatment with A1-R. As B16-OVA tumors became established at the 100-168 mm³ tumor size, greater than 90% of intratumoral SIINF-specific CD8⁺ T cells expressed a high level of PD-1 (FIG. 6). Treatment with A1-R control or A1-R SIINF did not reverse high PD-1 expression on the majority of SIINF-specific CD8⁺ T cells in the tumor. However, there was a non-significant trend suggesting that A1-R SIINF treatment may partially reduce the percentage of high PD-1-expressing SIINF-specific CD8⁺ T cells in the tumor (FIG. 6). In the same A1-R SIINF-treated mice, SIINF-specific CD8⁺ T cells from the spleen did not express a high level of PD-1 (FIG. 6). This demonstrated that high PD-1 expression was specific to SIINF-specific CD8⁺ T cells from the tumor and suggested a potential mechanism accounting for relapse following A1-R SIINF treatment.

### Example 6

### Treatment with Bacteria Expressing Tumor-Specific Peptide Synergizes with Anti-PD-L1 to Eradicate Long-Established Tumors. These Long-Established Tumors are Resistant to Anti-PD-L1 and Anti-CTLA-4

The following example shows that bacterial vaccination combined with anti-PD-L1 leads to consistent tumor eradication.

We tested whether the antitumor effects of A1-R SIINF could be enhanced by blocking the immunoinhibitory PD-1 and/or CTLA-4 pathway, which has also been implicated in suppressing T cell responses to tumors. B16-OVA tumors responded minimally to treatment with a combination of αPD-L1 and αCTLA-4 (FIG. 7A). Similarly, combining A1-R SIINF with αCTLA-4 was not effective as 0/4 tumors were rejected. However, combining A1-R SIINF with αPD-L1 or both αPD-L1 and αCTLA-4 was synergistic as 4/5 tumors were rejected in each group (FIG. 5A). Tumor rejection (4/5 mice) in each group was significant compared to the lack of rejection (0/5 mice) in the αCTLA-4 and αPD-L1 treatment group (p<0.05). Depleting CD8⁺ T cells in mice that rejected tumors did not result in relapse, strongly suggesting that treatment with A1-R SIINF combined with αPD-L1 resulted in complete tumor eradication (FIG. 7A).

To evaluate whether tumor rejection versus relapse was dependent on the magnitude of the SIINF-specific CD8⁺ T cell response, we measured the percentage of SIINF-specific CD8⁺ T cells in the blood of mice following the different treatments. The peripheral blood was a reliable indicator of the therapeutic response to A1-R SIINF treatment, since mice that rejected B16-OVA tumors had a significantly higher percentage of SIINF-specific CD8⁺ T cells compared to mice that had tumor relapse (FIG. 7B). Consistent with this finding, mice rejecting tumors following treatment with A1-R SIINF combined with αPD-L1 had a similar percentage of SIINF-specific CD8⁺ T cells as mice rejecting tumors following treatment with A1-R SIINF alone (FIG. 7B). These data demonstrate that the SIINF-specific CD8⁺ T cell response generated by A1-R SIINF can be enhanced by blocking PD-L1, most likely accounting for the consistent tumor eradication observed in these mice.

### Example 7

### Adoptively Transferred CD8 T Cells, with the Same T Cell Receptor Specificity as Generated by Bacterial Vaccination, Eradicated Tumors in 100% of Mice

The following example shows that adoptive transfer of T cells with this same vaccine-generated T cell receptor specificity results in eradication of long-established tumors. This suggests that vaccination can be used to identify T cell receptors that can be successfully used in adoptive T cell therapy protocols.

We evaluated whether adoptive transfer of SIINFEKL-specific CD8 T cells could eradicate long-established B16-OVA tumors. C57BL/6 mice bearing long-established B16-OVA tumors (day 18-24 post-inoculation) were treated with preconditioning irradiation (450 rad) followed or not by splenocytes from 1 naïve OT-1 mouse 24 later. OT-1 is a transgenic mouse with SIINFEKL-specific CD8 T cells. Irradiation alone had minimal effect on tumor progression. 7/7 tumors, from 2 independent experiments, were eradicated following adoptive transfer of OT-1 T cells.

### Example 8

### Discussion

To our knowledge, this is the first study reporting that therapeutic vaccination can rescue the dysfunctional endogenous tumor-specific CD8⁺ T cell response leading to eradication of long-established tumors. Treating mice with our antigen-producing S. Typhimurium A1-R vaccine rescued the tumor-specific CD8⁺ T cell response, most importantly with cytokine production recovered in the tumor. Treatment with our vaccine resulted in improved mouse survival and rejection of established tumors in approximately one-third of mice. We discovered that mice rejecting tumors had a significantly higher percentage of tumor-specific CD8⁺ T cells in their blood compared to mice that relapsed following treatment, suggesting that the magnitude of the generated antigen-specific CD8⁺ T cell response determined vaccine efficacy in our model. By combining our vaccine with αPD-L1, we enhanced the expansion of vaccine-generated CD8⁺ T cells and achieved consistent tumor rejection.

Our analysis focused on how to rescue tumor-specific CD8⁺ T cells specific to the SIINF epitope. By using two independent A1-R controls that did not produce SIINF, we determined that A1-R must produce SIINF in order to rescue the SIINF-specific CD8⁺ T cell response in the periphery and tumor. The requirement for A1-R to deliver SIINF strongly suggests that APCs mediating T cell rescue presented the SIINF epitope derived from A1-R rather than from B16 cancer cells. Since A1-R SIINF treatment induced systemic SIINF-specific CD8⁺ T cell proliferation, this suggests that A1-R delivered the SIINF epitope to APCs localized both within and outside the tumor. This is probable since A1-R SIINF persisted at a low level in normal organs following intravenous injection.

Rescuing CD8⁺ T cell dysfunction remains a significant challenge in cancer and chronic viral infection. Patients with the strongest responses to αPD-1 have tumors expressing PD-L1, which seems to be indicative of a pre-existing T cell response. However, the objective response rate for this PD-L1⁺ patient subset is still only 36%, suggesting that blocking PD-1/PD-L1 may be insufficient to rescue T cell dysfunction in many advanced tumors. In our model, long-established B16-OVA tumors were resistant to treatment with αPD-L1 and αCTLA-4 but were eradicated following treatment with A1-R SIINF combined with αPD-L1. While the exact mechanism accounting for synergy between A1-R SIINF and αPD-L1 will be further investigated, our data suggest that synergy may have occurred in both the lymphoid organs and tumor. A1-R SIINF-mediated expansion of SIINF-specific CD8⁺ T cells was enhanced by blocking PD-L1, suggesting that PD-L1 expression by APCs inhibited T cell expansion in the lymphoid organs. It is probable that A1-R treatment induced PD-L1 upregulation on APCs by inducing a strong inflammatory response and/or through a LPS-mediated mechanism. In A1-R SIINF-treated tumors, it is likely that SIINF-specific CD8⁺ T cell were inhibited by PD-1/PD-L1 interactions since (i) the majority of SIINF-specific CD8⁺ T cells expressed PD-1 at a high level and (ii) SIINF-specific CD8⁺ T cells had rescued capacity to produce IFN-γ, a potent stimulator of PD-L1 expression on B16 and human melanocytes in addition to stromal cells.

This study used B16-OVA to determine how to rescue tumor-specific CD8⁺ T cells in long-established tumors. We targeted a model tumor-specific antigen since targeting tumor-associated self antigens can lead to normal tissue damage and sometimes even patient death. It is probable from the above that the success of our vaccination approach relied on bacteria delivering exogenous tumor-specific peptide with high peptide-MHC affinity. High affinity mutant peptides can be identified by (i) whole-exome sequencing of cancer versus matched normal cells to identify somatic mutations followed by (ii) evaluating the affinity of mutant peptides using a p-MHC algorithm. We propose that this approach should be used to identify a mutant peptide or peptides that can be introduced into bacteria for therapeutic vaccination. Delivering multiple CD8⁺ T cell epitopes will likely prevent the relapse of tumors as ALVs. The ease in genetically-modifying bacteria to express different peptides makes this approach feasible.

Our approach to cancer vaccine development could also be used to rescue or induce T cells that are isolated from the patient and used for adoptive T cell therapy protocols. This could be especially beneficial for treating patients with larger tumor loads, since adoptive T cell therapy has shown great potential for treating large established tumors in experimental and clinical studies. As isolating sufficient numbers of functional tumor-specific T cells from patients remains a challenge, treating patients with our antigen-expressing bacterial vaccine combined with αPD-L1 prior to T cell isolation may significantly improve the quality of T cells that could be expanded in vitro and reinfused into patients. Vaccination could also be used to induce or rescue a tumor-specific CD8 T cell response for the purpose of (i) driving out a mutant tumor-specific CD8 T cell response, (ii) cloning the tumor-specific CD8 T cell, (iii) sequencing the T cell receptor that is specific to the tumor-specific peptide, (iv) transducing autologous T cells with the identified T cell receptor, and finally (v) re-infusing transduced T cells into the cancer patient. When we adoptively transferred CD8 T cells with the vaccine-generated T cell receptor specificity, we observed long-established tumors were rejected in 100% of mice. These data suggest the promise of using vaccination with mutant peptides that have high peptide-MHC affinity to improve adoptive T cell therapy protocols.

### Example 9

### Materials and Methods

### Cloning of Antigen Constructs and Verifying Antigen Expression

Antigen constructs were cloned into the pEGFP (Clontech, Mountain View, Calif.) plasmid. We codon optimized the OVA antigen construct (Invitrogen, Grand Island, N.Y.) encoding the first 104 amino acids of the SopE gene, the M45 epitope from the adenovirus E4-6/7 protein (30), and amino acids 248-357 of ovalbumin before inserting this antigen construct into the pEGFP backbone. Using standard cloning techniques, the SIINFEKL epitope of OVA was replaced by the irrelevant SNFVFAGI (31) epitope to make a control antigen construct. Expression plasmids were electroporated into A1-R bacteria. Antigen expression by A1-R was verified by western blot using an antibody against the M45 epitope (30) as previously described (16).

### Mice, Cell Lines, and Tumor Experiments

C57BL/6 and C57BL/6 CD8^{-/-} (B6.129S2-CD8a^{tm1Mak}/J) mice were purchased from the Jackson laboratory and maintained in a specific pathogen-free facility at the University of Chicago. Female mice were used at 8-14 weeks of age. All animal experimentation was conducted in accordance with the University of Chicago IACUC protocols.

The B16-OVA M04 cell line, a gift from Mary Jo Turk that was received in 2009, has been previously described (32). M04 was verified to express SIINFEKL using the 25-D1.16 antibody that recognizes the SIINFEKL peptide bound to H-2K^{b}. M04 consistently tested Mycoplama-free by the ATCC Universal Mycoplasma Detection kit (American Type Culture Collection, Manassas, Va.). Cancer cells were trypsinized, washed 1× in PBS, and injected at a dose of 5-10×10⁶ s.c. on the backs of mice. B16-OVA tumor take was >60% and all tumors that took invariably progressed rapidly and killed the host (20/20 tumors from 8 independent experiments). Tumor size was measured along three orthogonal axes (a, b, and c). Tumor volume=abc/2. Mice were sacrificed once tumors exceeded 1.5 cm³.

The leucine-arginine auxotrophic S. Typhimurium A1-R strain (33, 34) (AntiCancer, Inc., San Diego, Calif.) was intravenously injected at a dose of ˜2×10⁷ cfu in 200 μl 1×PBS per mouse. To study the effect of PD-L1 and CTLA-4 blockade, mice were treated with 150 μg of anti-PD-L1 (10F.9G2) and/or 100 μg of anti-CTLA-4 (UC10-4F10-11) i.p. every third day. Anti-CTLA-4 was purchased from the Fitch Monoclonal Facility (University of Chicago, Chicago, Ill.) and anti-PD-L1 was purchased from BioXcell (West Lebanon, N.H.). Further experimental details are in Supplemental Experimental Procedures.

### Flow Cytometry

A staining solution, referred to as SIINF-dX, containing SIINFEKL peptide-loaded K^{b}-DimerX [(K^{b})₂-IgG], Streptavidin-PE or -APC (BD Biosciences, San Diego, Calif.), and mouse IgG1 isotype control was used to detect SIINF-specific CD8⁺ T cells. A staining solution, referred to as SIYR-dX, was loaded with the irrelevant SIYRYYGL peptide and used as a control. Details regarding other antibodies and flow cytometric analyses are in the Supplemental Experimental Procedures.

### Peptide Restimulation In Vitro

Single cell suspensions from the tumor were used in a peptide restimulation assay. Cells were placed into a 96 well plate with each well containing 1-2×10⁶ viable cells in 200 μl RPMI with 20 μg/ml Brefeldin A, and 1 μg/ml SIINFEKL peptide (generously provided by S. Meredith, University of Chicago, Chicago, Ill.). Cells were restimulated with peptide for 5 hours at 37° C. Experimental details are provided in Supplemental Experimental Procedures.

### Statistical Analysis

A Mann-Whitney test was used to perform pairwise comparisons of tumor sizes between different groups during outgrowth. Both nonparametric Kruskal-Wallis and parametric analysis of variance (followed by Sidak's multiple comparisons procedure) were performed for analysis of 3 or more groups. Fisher's exact test was used to compare survival rates of mice at an indicated time or tumor rejection rates between different treatment groups. Wilcoxon-signed rank and paired T tests following parametric transformation of the data were used to compare the percentage of CD8⁺ T cells that were stained by SIINF-dX or SIYR-dX. The P values reported in this manuscript were derived from parametric analysis. All results reported to be significant following parametric analysis were also significant or at least marginally significant (p<0.06) using the nonparametric tests.

### In Vitro S. Typhimurium A1-R Antigen Delivery Assay

J774 K^{b}-expressing macrophages, a gift from Chyung-Ru Wang (Northwestern University, Chicago, Ill.), were plated overnight at 5×10⁴ cells per well in a 96 well plate. A1-R control or A1-R SIINF were grown as described above, washed 2× in PBS, and added to the macrophages in 200 μl of RPMI at an MOI of either 20 or 160 bacteria per macrophage. Macrophages were infected with bacteria for 75 minutes at 37° C. in a 10% dry incubator, washed 1× in PBS, and then incubated with 100 μg/ml gentamicin for 1 hour to kill the extracellular bacteria. Macrophages were subsequently washed 2× in RPMI and 1×10⁵ B3Z hybridoma cells were added to each well in 200 μl of RPMI containing 5% FCS, 20 mM L-Glutamine, and 100 μg/ml gentamicin. After a 24 hour incubation at 37° C. in a 5% CO₂ humidified incubator, culture supernatants were harvested and an IL-2 ELISA was conducted to determine the extent of B3Z stimulation by infected macrophages.

S. Typhimurium A1-R Preparation and Injection

The leucine-arginine auxotrophic S. Typhimurium A1-R strain (AntiCancer, Inc., San Diego, Calif.) has been previously described (33, 34). A1-R was grown overnight in 10 ml of Miller's LB Broth Base (Invitrogen, Grand Island, N.Y.) in a 14 ml polypropylene round-bottom tube (BD, San Diego, Calif.) by shaking at 220 rpm at 37° C. On the next day, overnight cultures were diluted 1:20 and grown for approximately 4 additional hours by shaking at 37° C. until the OD₆₀₀ equaled 0.5-0.6 (approximately 5-6×10⁸ bacterium/ml); the bacteria were washed 2 times in 1×PBS and intravenously injected at a dose of ˜2×10⁷ cfu in 200 μl 1×PBS per mouse.

### Flow Cytometry

Cells were stained using FITC-, PE-, PerCP-, or APC-labeled mAb directed against mouse CD8a (53-6.7), PD-1 (J43), TNF-α (MP6-XT22), IFN-γ (XMG1.2), and SIINFEKL peptide bound to H-2K^{b} (25-D1.16). Mouse IgG1 (RMG1-1) and Armenian hamster IgG (eBio299Arm) were used as isotype controls. All antibodies and staining solutions were purchased from BD or eBioscience (San Diego, Calif.). Flow cytometry data was recorded on FACSCalibur or FACSCANTO machines (BD). Data were analyzed using FlowJo (Tree Star, Ashland, Oreg.) software.

Preparation of Single-Cell Suspensions from Tissues and Peptide Restimulation In Vitro

Tumor fragments were incubated with 1 mg/ml collagenase D (Roche, Mannheim, Germany) and 0.25 mg/ml DNAse I (Roche) in 5 ml RPMI media for 30 minutes at 37° C. in a 5% CO₂ humidified incubator. 500 μl of 0.25% trypsin (Invitrogen, Grand Island, N.Y.) was added to the cell suspension which was continuously pippeted for 2 minutes to break clusters. RPMI containing 5% FCS (Sigma-Aldrich, St. Louis, Mo.) was added to neutralize the trypsin, and the suspension was filtered through a 70 μm nylon filter mesh. The tumor single cell suspension was then stained for flow cytometry or used in a peptide restimulation assay. For peptide restimulation, cell suspensions were stained with SIINF-dX or SIYR-dX (BD Biosciences, San Diego, Calif.) for 20 minutes at 4° C. and then placed into a 96 well plate with each well containing 1-2×10⁶ viable cells in 200 μl RPMI with 10% FCS, 2 mmol/I glutamine, 50 μmol/β-mercaptoethanol, 1 mmol/1 Hepes, 1 mmol/l sodium pyruvate, 1× nonessential amino acids, 20 μg/ml Brefeldin A, and 1 μg/ml SIINFEKL peptide (generously provided by S. Meredith, University of Chicago, Chicago, Ill.). Cells were restimulated with peptide for 5 hours at 37° C. in a 5% CO₂ humidified incubator.

CD8⁺ T Cell Depletion

For CD8⁺ T cell depletion following A1-R SIINF treatment, anti-CD8 (YTS-169.4) was injected for the first time at 4 days post-A1-R SIINF treatment. Mice were injected i.p. with 200 μg of anti-CD8 every third day until tumors reached 1.5 cm³. When depleting CD8⁺ T cells to determine if B16-OVA tumors were fully eradicated, mice were injected with 200 μg of anti-CD8 i.p: every third day for a total of 3 doses and tumor relapse was monitored for at least 30 days. CD8⁺ T cell depletion was confirmed by flow cytometry. Anti-CD8 was purchased from the Fitch Monoclonal Facility (University of Chicago, Chicago, Ill.).

### Adoptive T Cell Transfer

C57BL/6 mice bearing established B16OVA tumors (day 18-24) were treated with preconditioning irradiation (450 rad) followed or not by splenocytes from 1 naïve OT1 mouse 24 later.

### Other Embodiments

While the invention has been described in conjunction with the detailed description thereof, the foregoing description is intended to illustrate and not limit the scope of the invention, which is defined by the scope of claims. Other aspects, advantages, and modifications are within the scope of the following claims.

1. A methodology, using vaccination with tumor-specific peptides that have high peptide-MHC affinity (low nanomolar or sub-nanomolar affinity) to overcome tumor resistance to immunostimulatory blocking antibodies.

2. A methodology, using cancer vaccination with tumor-specific peptides to induce or rescue tumor-specific T cells that can be used in adoptive T cell therapy protocols.

3. A methodology, using cancer vaccination with tumor-specific peptides to identify T cell receptor sequences that can be transduced into autologous lymphocytes as part of adoptive T cell therapy protocols.

4. The method of claims 1-3, wherein vaccination can be combined with immunostimulatory monoclonal antibodies.
